(19) United States
(12) Patent Application Publication
(10) Pub. No.: US 2004/0221335 A1
(43) Pub. Date: Nov. 4, 2004

(54) Method for Increasing Total Oil Levels in Plants

(75) Inventors: Christine K. Shewmaker, Woodland, CA (US); Alison Van Eenennaam, Davis, CA (US); Deborah J. Hawkins, Davis, CA (US); Rick A. Sanders, Davis, CA (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(73) Assignee: MONSANTO TECHNOLOGY LLC, St. Louis, MO

(21) Appl. No.: 10/604,708

(22) Filed: Aug. 12, 2003

Related U.S. Application Data
(60) Provisional application No. 60/402,527, filed on Aug. 12, 2002.

(51) Int. Cl. A01H 1/00

(52) U.S. Cl. 800/281

(57) ABSTRACT

The present invention is in the field of plant genetics and biochemistry. More specifically, the present invention relates to genes affecting the level and composition of oil in plants. In particular, the present invention is directed to methods for increasing the oil level in plants and seeds. Moreover, the present invention includes and provides methods for producing plants and obtaining seeds with altered fatty acid composition.

This application claims priority to U.S. provisional application 60/402,527 filed on Aug. 12, 2002, herein incorporated by reference in its entirety.

### BACKGROUND OF INVENTION

The present invention is in the field of plant genetics and biochemistry. More specifically, the present invention relates to the level of total oil in plants. In particular, the present invention is directed to methods for increasing the oil level and altering the oil composition in plants and seeds. Moreover, the present invention includes and provides methods for producing plants and obtaining seed with increased oil levels. Such plants and seeds can also exhibit essentially unaltered protein compositions.

Plant oils are utilized in a wide variety of applications. For example, soybean oils have been used in applications as diverse as salad and cooking oils to biodiesel and biolube oils. Seed oils are composed almost entirely of triacylglycerols in which fatty acids are esterified to each of the three hydroxyl groups of glycerol. The use of triacylglycerols as a seed reserve maximizes the quantity of stored energy within a limited volume, because the fatty acids are a highly reduced form of carbon (Miquel and Browse, in Seed Development and Germination, Galili et al. (eds.), Marcel Dekker, New York, pp. 169-193, 1994). A large variety of different fatty acid structures are found in nature (Gunstone et al., The Lipid Handbook, Chapman & Hall, London, 1994; Hilditch and Williams, The Chemical Constituents of Natural Fats, Chapman & Hall, London, 1964; Murphy, Designer Oil Crops, VCH, Weinheim, 1994; van de Loo et al., Proc. Natl Acad. Sci. USA, 92:6743-6747, 1993), but just five account for 90% of the commercial vegetable oil produced: palmitic (16:0), stearic (18:0), oleic (18:1), linoleic (18:2), and α-linolenic (18:3) acid.

Factors governing the total oil level of a plant or plant part such as a seed are complex. As such, selection for increased total oil is often a laborious process often with the resulting plants exhibiting considerable plant-to-plant variation (Jensen, Plant Breeding Methodology, John Wiley & Sons, Inc., USA, 1988). Moreover, selection for increased total oil often results in a decrease in the protein fraction of the seed. Thus, there remains a need for methods of producing plants with increased total oil, particularly a method that also produces plants with essentially unaltered protein levels.

### SUMMARY OF INVENTION

The present invention includes and provides a method for increasing total oil level in a seed comprising: (A) transforming a plant with a nucleic acid construct that comprises as operably linked components, a promoter, a structural nucleic acid sequence capable of modulating the level of FAD2 mRNA or FAD2 protein; and (B) growing the plant.

The present invention includes and provides a method for increasing total oil in a seed comprising: (A) transforming a plant with a nucleic acid construct that comprises as operably linked components, a promoter, a structural nucleic acid sequence capable of increasing the level of oleic acid; and (B) growing the plant.

The present invention includes and provides a method of obtaining a seed having increased total oil level comprising: (A) growing a plant having a modulated level of a FAD2 protein or a FAD2 mRNA; and (B) obtaining the seed from the plant.

The present invention includes and provides a method for increasing percentage of total oil in a seed comprising: (A) transforming a plant with a nucleic acid construct that comprises as operably linked components, a promoter, a structural nucleic acid sequence capable of modulating the level of FAD2 mRNA or FAD2 protein; and (B) growing the plant.

The present invention includes and provides a method for the production of a plant having an increased percentage of total oil comprising: (A) crossing a first plant having a modified level of a FAD2 protein or a FAD2 mRNA with a second plant to produce a segregating population; (B) screening the segregating population for a member having an increased percentage of total oil; and (C) selecting the member.

The present invention includes and provides chimeric genes comprising an isolated nucleic acid fragment encoding a delta-12 desaturase or any functionally equivalent subfragment or the reverse complement of such fragment or subfragment that are operably linked and wherein expression of such combinations results in an increase in total oil.

Also included in this invention are plants and plant parts thereof containing the various chimeric genes, seeds of such plants, oil obtained from the grain of such plants, animal feed derived from the processing of such grain, the use of the foregoing oil in food, animal feed, cooking oil or industrial applications, products made from the hydrogenation, fractionation, interesterification or hydrolysis of such oil and methods for improving the carcass quality of an animal.

### BRIEF DESCRIPTION OF DRAWINGS

FIG. 1 depicts the construct pMON67563.

FIG. 2 depicts a correlation of percentage of total oil versus oleic acid (18:1) in pMON67563 and pCGN9979 control lines.

FIG. 3 depicts oleic acid (18:1) level versus percentage of total oil in Arabidopsis seed.

FIG. 4 depicts mean (SEM) oil percentage in T₃ seed from transgenic lines expressing the FAD2 dsRNAi suppression construct (right) versus control lines containing an empty vector (left).

FIG. 5 depicts the construct pMON67589.

FIG. 6 depicts the construct pMON67591.

FIG. 7 depicts the construct pMON67592.

FIG. 8 depicts the construct pMON68655.

FIG. 9 depicts the construct pMON68656.

### DETAILED DESCRIPTION

Definitions

As used herein, “total oil level” refers to the total aggregate amount of fatty acid without regard to the type of fatty acid.

As used herein, the term “gene” is used to refer to the nucleic acid sequence that encompasses the 5′ promoter region associated with the expression of the gene product, any intron and exon regions and 3′ untranslated regions associated with the expression of the gene product.

As used herein, a “FAD2”, “Δ12 desaturase” or “omega-6 desaturase” is an enzyme capable of catalyzing the insertion of a double bond into a fatty acyl moiety at the twelfth position counted from the carboxyl terminus.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant. Chimeric genes can be designed for use in cosuppression or antisense by linking a nucleic acid fragment or subfragment thereof, whether or not it encodes an active enzyme, in the appropriate orientation relative to a plant promoter sequence.

The term “non-coding” refers to sequences of nucleic acid molecules that do not encode part or all of an expressed protein. Non-coding sequences include but are not limited to introns, promoter regions, 3′ untranslated regions, and 5′ untranslated regions.

The term “intron” as used herein refers to the normal sense of the term as meaning a segment of nucleic acid molecules, usually DNA, that does not encode part of or all of an expressed protein, and which, in endogenous conditions, is transcribed into RNA molecules, but which is spliced out of the endogenous RNA before the RNA is translated into a protein.

The term “exon” as used herein refers to the normal sense of the term as meaning a segment of nucleic acid molecules, usually DNA, that encodes part of or all of an expressed protein.

As used herein, when referring to proteins and nucleic acids herein, the use of plain capitals, e.g., “FAD2”, indicates a reference to an enzyme, protein, polypeptide, or peptide, and the use of italicized capitals, e.g., “FAD2”, is used to refer to nucleic acids, including without limitation genes, cDNAs, and mRNAs.

As used herein, a promoter that is “operably linked” to one or more nucleic acid sequences is capable of driving expression of one or more nucleic acid sequences, including multiple coding or non-coding nucleic acid sequences arranged in a polycistronic configuration.

As used herein, the term complement of a nucleic acid sequence refers to the complement of the sequence along its complete length.

As used herein, any range set forth is inclusive of the end points of the range unless otherwise stated.

One skilled in the art may refer to general reference texts for detailed descriptions of known techniques discussed herein or equivalent techniques. These texts include Ausubel et al., Current Protocols in Molecular Biology, John Wiley and Sons, Inc., 1995; Sambrook et al., Molecular Cloning, A Laboratory Manual (2d ed.), Cold Spring Harbor Press, Cold Spring Harbor, N.Y., 1989; Birren et al., Genome Analysis: A Laboratory Manual, volumes 1 through 4, Cold Spring Harbor Press, Cold Spring Harbor, N.Y., 1997-1999; Plant Molecular Biology: A Laboratory Manual, Clark (ed.), Springer, N.Y., 1997; Richards et al., Plant Breeding Systems (2d ed.), Chapman & Hall, The University Press, Cambridge, 1997; and Maliga et al., Methods in Plant Molecular Biology, Cold Spring Harbor Press, Cold Spring Harbor, N.Y., 1995. These texts can, of course, also be referred to in practicing an aspect of the invention.

The present invention includes and provides a method for increasing total oil level in a seed comprising: (A) transforming a plant with a nucleic acid construct that comprises as operably linked components, a promoter, a structural nucleic acid sequence capable of modulating the level of FAD2 mRNA or FAD2 protein; and (B) growing the plant. The structural nucleic acid sequence can be selected from the group of SEQ ID NOS: 1, 4, 7-11, 14, 19, 22, 25 or 26 or the reverse complement thereof, any functionally equivalent subfragment thereof or the reverse complement of said fragment or subfragment.

The present invention provides a method for increasing total oil level in a seed. An increase of total oil can be an increase of any amount. An increase of total oil may result from altering the level of any enzyme or transcript that increases oleic acid level (18:1). In a preferred aspect, an increase in total oil is the percentage increase between the total oil found in a seed or collection of seeds and the total oil measured in a second or subsequent seed or collection of seeds. As used herein, percentage increase is calculated as the difference between the total oil found in a seed or collection of seeds and the total oil measured in a second or subsequent seed or collection of seeds. In a particularly preferred aspect, the increase in total oil is measured relative to a seed from a plant with a similar genetic background but lacking a structural nucleic acid sequence capable of affecting the level of oleic acid (18:1). In another particularly preferred aspect, the increase in total oil is measured relative to a seed from a plant with a similar genetic background but lacking a structural nucleic acid sequence capable of modulating the level of FAD2 mRNA or FAD2 protein.

When levels of an agent are compared, such a comparison is preferably carried out between organisms with a similar genetic background. In a preferred aspect, a similar genetic background is a background where the organisms being compared share 50% or greater of their nuclear genetic material. In a more preferred aspect a similar genetic background is a background where the organisms being compared share 75% or greater, even more preferably 90% or greater of their nuclear genetic material. In another even more preferable aspect, a similar genetic background is a background where the organisms being compared are plants, and the plants are isogenic except for any genetic material originally introduced using plant transformation techniques.

In another aspect, the increase is measured in a seed of a plant produced by crossing two plants and the increase in a seed of that plant is measured relative to one or more of the seeds of one or more of the plants utilized to generate the plant in question (i.e., parents).

Total oil levels can be measured by any appropriate method. For example, without limitation, quantitation of oil content of seeds is often performed with conventional methods, such as near infrared analysis (NIR), nuclear magnetic resonance imaging (NMR), soxhlet extraction, accelerated solvent extraction (ASE), microwave extraction, and super critical fluid extraction. Near infrared (NIR) spectroscopy has become a standard method for screening seed samples whenever the sample of interest has been amenable to this technique. Samples studied include wheat, maize, soybean, canola, rice, alfalfa, oat, and others.

NIR analysis of single seeds can be used (see Velasco et al., “Estimation of Seed Weight, Oil Content and Fatty Acid Composition in Intact Single Seeds of Rapeseed (Brassica napus L.) by Near-Infrared Reflectance Spectroscopy,” Euphytica, Vol. 106, 1999, pp. 79-85; Delwiche, “Single Wheat Kernel Analysis by Near-Infrared Transmittance: Protein Content,” Analytical Techniques and Instrumentation, Vol. 72, 1995, pp. 11-16; Dowell, “Automated Color Classification of Single Wheat Kernels Using Visible and Near-Infrared Reflectance,” Vol. 75(1), 1998, pp. 142-144; Dowell et al., “Automated Single Wheat Kernel Quality Measurement Using Near-Infrared Reflectance,” ASAE Annual International Meeting, 1997, paper number 973022, all of which are herein incorporated by reference in their entirety). NMR has also been used to analyze oil content in seeds (see, for example, Robertson and Morrison, “Analysis of Oil Content of Sunflower Seed by Wide-Line NMR,” Journal of the American Oil Chemists Society, 1979, Vol. 56, 1979, pp. 961-964, which is herein incorporated by reference in its entirety).

Other techniques, including soxhlet extraction, accelerated solvent extraction (ASE), microwave extraction, and super critical fluid extraction, can be used to determine oil content. Some techniques use gravimetry as the final measurement step (see, for example, Taylor et al, “Determination of Oil Content in Oilseeds by Analytical Super-critical Fluid Extraction,” Vol. 70 (No. 4), 1993, pp. 437-439, which is herein incorporated by reference in its entirety). Gravimetry, however, is not suitable for use with small samples, including small seeds and seed with little oil content, because oil levels in these samples can be below the level of minimum sensitivity for the technique. Furthermore, the use of gravimetry is time consuming and is not amenable to high-throughput automation.

The methods of the present invention may be used to increase total oil level in any seed. In a preferred embodiment, a seed includes either endosperm or embryo. In another preferred embodiment, a seed includes both endosperm and embryo. The seeds can be from either dicots or monocots. In a preferred embodiment, the seed may be selected from the group consisting of Arabidopsis seed, Brassica seed, canola seed, corn seed, oil palm seed, oilseed rape seed, peanut seed, rapeseed seed, safflower seed, soybean seed, and sunflower seed, with Arabidopsis seed, Brassica seed, canola seed, corn seed, and soybean seed particularly preferred.

Transforming a plant may be effected by any means that results in the introduction of a construct into a plant. Various methods for the introduction of a desired polynucleotide sequence into plant cells are available and known to those of skill in the art and include, but are not limited to: (1) physical methods such as microinjection, electro-poration, and microprojectile mediated delivery (biolistics or gene gun technology); (2) virus mediated delivery methods; and (3) Agrobacterium-mediated transformation methods.

The most commonly used methods for transformation of plant cells are the Agrobacterium-mediated DNA transfer process and the biolistics or microprojectile bombardment mediated process (i.e., the gene gun). Typically, nuclear transformation is desired but where it is desirable to specifically transform plastids, such as chloroplasts or amyloplasts, plant plastids may be transformed utilizing a microprojectile mediated delivery of the desired polynucleotide.

Agrobacterium-mediated transformation is achieved through the use of a genetically engineered soil bacterium belonging to the genus Agrobacterium. A number of wild-type and disarmed strains of Agrobacterium tumefaciens and Agrobacterium rhizogenes harboring Ti or Ri plasmids can be used for gene transfer into plants. Gene transfer is done via the transfer of a specific DNA known as “T-DNA”, that can be genetically engineered to carry any desired piece of DNA into many plant species.

Agrobacterium-mediated genetic transformation of plants involves several steps. The first step, in which the virulent Agrobacterium and plant cells are first brought into contact with each other, is generally called “inoculation”. Following the inoculation, the Agrobacterium and plant cells/tissues are permitted to be grown together for a period of several hours to several days or more under conditions suitable for growth and T-DNA transfer. This step is termed “co-culture”. Following co-culture and T-DNA delivery, the plant cells are treated with bactericidal or bacteriostatic agents to kill the Agrobacterium remaining in contact with the explant and/or in the vessel containing the explant. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is typically followed by one or more “selection” steps.

With respect to microprojectile bombardment (U.S. Pat. No. 5,550,318; U.S. Pat. No. 5,538,880; U.S. Pat. No. 5,610,042; and PCT Publication WO 95/06128; each of which is specifically incorporated herein by reference in its entirety), particles are coated with nucleic acids and delivered into cells by a propelling force. Exemplary particles include those comprised of tungsten, platinum, and preferably, gold.

An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with plant cells cultured in suspension.

Microprojectile bombardment techniques are widely applicable and may be used to transform virtually any plant species. Examples of species that have been transformed by microprojectile bombardment include monocot species such as maize (PCT Publication WO 95/06128), barley, wheat (U.S. Pat. No. 5,563,055, specifically incorporated herein by reference in its entirety), rice, oat, rye, sugarcane, and sorghum; as well as a number of dicots including tobacco, soybean (U.S. Pat. No. 5,322,783, specifically incorporated herein by reference in its entirety), sunflower, peanut, cotton, tomato, and legumes in general (U.S. Pat. No. 5,563,055, specifically incorporated herein by reference in its entirety).

To select or score for transformed plant cells regardless of transformation methodology, the DNA introduced into the cell may contain a gene that functions in a regenerable plant tissue to produce a compound that confers upon the plant tissue resistance to an otherwise toxic compound. Genes of interest for use as a selectable, screenable, or scorable marker would include but are not limited to GUS, green fluorescent protein (GFP), luciferase (LUX), antibiotic or herbicide tolerance genes. Examples of antibiotic resistance genes include the penicillins, kanamycin (and neomycin, G418, bleomycin); methotrexate (and trimethoprim); chloramphenicol; kanamycin and tetracycline. The regeneration, development, and cultivation of plants from various transformed explants is well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. Developing plantlets are transferred to soil-less plant growth mix, and hardened off, prior to transfer to a greenhouse or growth chamber for maturation.

The present invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Any suitable plant culture medium can be used. Examples of suitable media would include but are not limited to MS-based media (Murashige and Skoog, Physiol. Plant, 15:473-497, 1962) or N6-based media(Chu et al., Scientia Sinica 18:659, 1975) supplemented with additional plant growth regulators including but not limited to auxins, cytokinins, ABA, and gibberellins. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Those of skill in the art are aware that media and media supplements such as nutrients and growth regulators for use in transformation and regeneration and other culture conditions such as light intensity during incubation, pH, and incubation temperatures that can be optimized for the particular variety of interest.

A construct or vector may include a plant promoter to express the nucleic acid molecule of choice. In a preferred embodiment, any nucleic acid molecules described herein can be operably linked to a promoter region that functions in a plant cell to cause the production of an mRNA molecule. For example, any promoter that functions in a plant cell to cause the production of an mRNA molecule, such as those promoters described herein, without limitation, can be used. In a preferred embodiment, the promoter is a plant promoter.

A number of promoters that are active in plant cells have been described in the literature. These include, but are not limited to, the nopaline synthase (NOS) promoter (Ebert et al., Proc. Natl. Acad. Sci. (U.S.A.) 84:5745-5749, 1987), the octopine synthase (OCS) promoter (which is carried on tumor-Inducing plasmids of Agrobacterium tumefaciens), the caulimovirus promoters such as the cauliflower mosaic virus (CaMV) 19S promoter (Lawton et al., Plant Mol. Biol. 9:315-324, 1987) and the CaMV 35S promoter (Odell et al., Nature 313:810-812, 1985), the figwort mosaic virus 35S-promoter (U.S. Pat. No. 5,378,619), the light-Inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628, 1987), the sucrose synthase promoter (Yang et al., Proc. Natl. Acad. Sci. (U.S.A.) 87:4144-4148, 1990), the R gene complex promoter (Chandler et al., The Plant Cell 1:1175-1183, 1989) and the chlorophyll a/b binding protein gene promoter. These promoters have been used to create DNA constructs that have been expressed in plants; see, e.g., PCT publication WO 84/02913. The CaMV 35S promoters are preferred for use in plants. Promoters known or found to cause transcription of DNA in plant cells can be used in the invention.

Other promoters can also be used to express a polypeptide in specific tissues, such as seeds or fruits. Indeed, in a preferred embodiment, the promoter used is a seed-specific promoter. Examples of such promoters include the 5′ regulatory regions from such genes as napin (Kridl et al, Seed Sci. Res. 1:209:219, 1991), phaseolin (Bustos et al, Plant Cell, 1(9):839-853, 1989), soybean trypsin inhibitor (Riggs et al, Plant Cell 1(6):609-621, 1989), ACP (Baerson et al., Plant Mol. Biol., 22(2):255-267, 1993), stearoyl-ACP desaturase (Slocombe et al., Plant Physiol. 104(4):167-176, 1994), soybean a′ subunit of β-conglycinin (P-Gm7S, see for example, Chen et al., Proc. Natl. Acad. Sci. 83:8560-8564, 1986), Vicia faba USP (P-Vf.Usp, see for example, SEQ ID NO: 1, 2, and 3 in U.S. patent application Ser. No. 10/429,516) and Zea mays L3 oleosin promoter (P-Zm.L3, see, for example, Hong et al., Plant Mol. Biol., 34(3):549-555, 1997). Also included are the zeins, which are a group of storage proteins found in corn endosperm. Genomic clones for zein genes have been isolated (Pedersen et al., Cell 29:1015-1026, 1982; and Russell et al., Transgenic Res. 6(2):157-168) and the promoters from these clones, including the 15 kD, 16 kD, 19 kD, 22 kD, 27 kD and genes, could also be used. Other promoters known to function, for example, in corn include the promoters for the following genes: waxy, Brittle, Shrunken 2, Branching enzymes I and II, starch synthases, debranching enzymes, oleosins, glutelins and sucrose synthases. A particularly preferred promoter for corn endosperm expression is the promoter for the glutelin gene from rice, more particularly the Osgt-1 promoter (Zheng et al., Mol. Cell Biol. 13:5829-5842, 1993). Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins and the glutenins. Examples of such promoters in rice include those promoters for the ADPGPP subunits, the granule bound and other starch synthase, the branching enzymes, the debranching enzymes, sucrose synthases and the glutelins. A particularly preferred promoter is the promoter for rice glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins. A preferred promoter for expression in the seed is a napin promoter, referred to herein as P-Br.Snap2. Another preferred promoter for expression is an Arcelin5 promoter (U.S. Patent Publication 2003/0046727). Yet another preferred promoter is a soybean 7S promoter (P-Gm.7S) and the soybean 7Sα″ beta conglycinin promoter (P-Gm.Sphas1).

Additional promoters that may be utilized are described, for example, in U.S. Pat. No. 5,378,619; 5,391,725; 5,428,147; 5,447,858; 5,608,144; 5,608,144; 5,614,399; 5,633,441; 5,633,435; and 4,633,436. In addition, a tissue specific enhancer may be used.

Constructs or vectors may also include, with the region of interest, a nucleic acid sequence that acts, in whole or in part, to terminate transcription of that region. A number of such sequences have been isolated, including the Tr7 3″ sequence and the NOS 3″ sequence (Ingelbrecht et al., The Plant Cell 1:671-680, 1989; Bevan et al., Nucleic Acids Res. 11:369-385, 1983). Regulatory transcript termination regions can be provided in plant expression constructs of this invention as well. Transcript termination regions can be provided by the DNA sequence encoding the gene of interest or a convenient transcription termination region derived from a different gene source, for example, the transcript termination region that is naturally associated with the transcript initiation region. The skilled artisan will recognize that any convenient transcript termination region that is capable of terminating transcription in a plant cell can be employed in the constructs of the present invention.

A vector or construct may also include regulatory elements. Examples of such include the Adh intron 1 (Callis et al., Genes and Develop. 1:1183-1200, 1987), the sucrose synthase intron (Vasil et al., Plant Physiol. 91:1575-1579, 1989) and the TMV omega element (Gallie et al., The Plant Cell 1:301-311, 1989). These and other regulatory elements may be included when appropriate.

It is understood that two or more nucleic acid molecules of the present invention may be introduced into a plant using a single construct and that construct can contain one or more promoters. In embodiments where the construct is designed to express two nucleic acid molecules, it is preferred that the two promoters are (i)constitutive promoters, (ii)seed-specific promoters, or (iii)constitutive promoter and one seed-specific promoter. Preferred seed-specific promoters are 7S, napin, and maize globulin-1 gene promoters. A preferred constitutive promoter is a CaMV promoter. It is further understood that two or more of the nucleic molecules may be physically linked and expressed utilizing a single promoter, preferably a seed-specific or constitutive promoter.

In a preferred embodiment of the present invention, post-transcriptional gene silencing may be induced in plants by transforming them with antisense or co-suppression constructs. In particular, constructs constructed by the methods of Smith et al. (Nature 407: 319-320, 2000) may be used to good effect. Other methods of construction are well known to one of skill in the art and have been reviewed.

Structural nucleic acid sequences capable of decreasing the level of FAD2 mRNA or FAD2 protein include any nucleic acid sequence with sufficient homology to FAD2 gene. Exemplary nucleic acids include those set forth in U.S. Pat. No. 6,372,965, U.S. Pat. No. 6,342,658, U.S. Pat. No. 6,333,448, U.S. Pat. No. 6,291,741, U.S. Pat. No. 6,063,947, WO 01/14538 A3, U.S. PAP 2002/20058340, and U.S. PAP 2002/0045232.

The present invention includes and provides a method for the production of a plant having increased total oil level as compared to at least one of a first or a second plant comprising: (A) crossing a first plant having a modified level of a FAD2 protein or a FAD2 mRNA with a second plant to produce a segregating population; (B) screening the segregating population for a member having the modified level of a FAD2 protein or a FAD2 mRNA; and (C) selecting the member.

The present invention includes and provides a method for the production of a plant having an increased percentage of total oil comprising: (A) crossing a first plant having a modified level of a FAD2 protein or a FAD2 mRNA with a second plant to produce a segregating population; (B) screening the segregating population for a member having an increase in total oil; and (C) selecting the member.

The present invention includes and provides a method for the production of a plant having an increased percentage of total oil comprising: (A) crossing a first plant having an increased level of oleic acid and a decreased level of linoleic acid with a second plant to produce a segregating population; (B) screening the segregating population for a member having the increased level of oleic acid and the decreased level of linoleic acid; and (C) selecting the member.

Plants of the present invention can be part of or generated from a breeding program. The choice of breeding method depends on the mode of plant reproduction, the heritability of the trait(s) being improved, and the type of cultivar used commercially (e.g., F₁ hybrid cultivar, pureline cultivar, etc). Selected, non-limiting approaches, for breeding the plants of the present invention are set forth below. A breeding program can be increased using marker assisted selection of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment, a backcross or recurrent breeding program is undertaken. The complexity of inheritance influences the choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new cultivars requires the development and selection of varieties, the crossing of these varieties and the selection of superior hybrid crosses. The hybrid seed can be produced by manual crosses between selected male-fertile parents or by using male sterility systems. Hybrids are selected for certain single gene traits such as pod color, flower color, seed yield, pubescence color, or herbicide resistance, which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits are crossed to produce an F₁ An F₂ population is produced by selfing one or several F₁'s. Selection of the best individuals from the best families is carried out. Replicated testing of families can begin in the F₄ generation to improve the effectiveness of selection for traits with low heritability. At an advanced stage of in-breeding (i.e., F₆ and F₇), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of in-breeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, breeders commonly harvest one or more pods from each plant in a population and thresh them together to form a bulk. Part of the bulk is used to plant the next generation and part is put in reserve. The procedure has been referred to as modified single-seed descent or the pod-bulk technique. The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g., Fehr, Principles of Cultivar Development, Vol. 1, 1987).

A transgenic plant of the present invention may also be reproduced using apomixis. Apomixis is a genetically controlled method of reproduction in plants where the embryo is formed without union of an egg and a sperm. Apomixis is economically important, especially in transgenic plants, because it causes any genotype, no matter how heterozygous, to breed true. Thus, with apomictic reproduction, heterozygous transgenic plants can maintain their genetic fidelity throughout repeated life cycles. Methods for the production of apomictic plants are known in the art. See, e.g., U.S. Pat. No. 5,811,636.

All articles, patents, and patent applications cited herein are incorporated by reference in their entirety.

The following examples are illustrative and not intended to be limiting in anyway.

### EXAMPLES

### Example 1

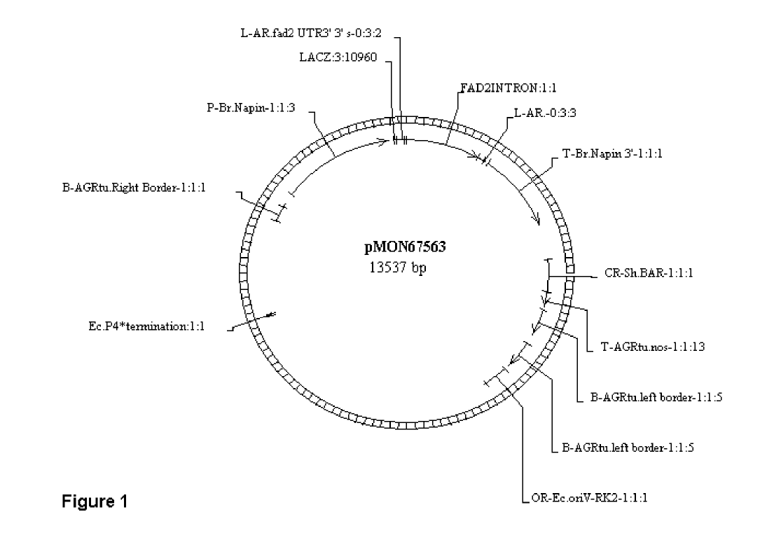

A gene silencing construct is produced according to the method of Smith et al. in order to reduce FAD2 expression in Arabidopsis through post transcriptional gene silencing (PTGS). (Smith et al., Nature 407: 319-320, 2000). A construct (pMON67563, FIG. 1) is constructed using the napin promoter to drive expression of a hairpin RNA (hpRNA) containing 120 nucleotides of the 3″-untranslated region of FAD2 in sense (SEQ ID NO: 1) and antisense orientation flanking an intron. Arabidopsis plants are transformed with pMON67563 by Agrobacterium-mediated transformation. An empty napin vector (pCGN9979) is also transformed into Arabidopsis plants by Agrobacterium-mediated transformation as a control.

### Example 2

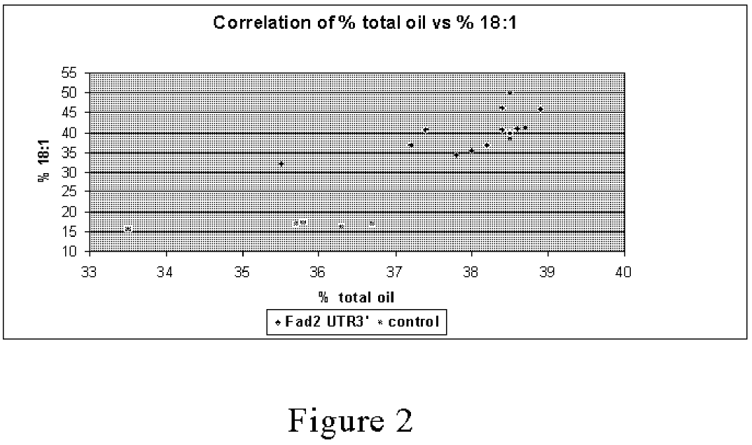

Seed from transformed Arabidopsis plants is analyzed by gas chromatography (GC) and near infrared spectroscopy (NIR) for fatty acid profile and total oil content. GC analysis demonstrates that Arabidopsis plants transformed with pMON67563 have an increased proportion of oleic acid (18:1) and a decreased proportion of linoleic acid (18:2) relative to controls. Transformed strains 67563-1 through 67563-13 show an increased proportion of oleic acid (18:1) and a decreased proportion of linoleic acid (18:2) relative to untransformed control strains 9979-11 through 9979-15. The relative amounts of oleic acid and linoleic acid are measured in percent (w/w) with control strains 9979-11 through 9979-15 exhibiting an oleic acid level ranging between about 14% (w/w) and about 18% (w/w) and a linoleic acid level ranging between about 30% (w/w) and about 32% (w/w). Transformed strains 67563-1 through 67563-3 and 67563-5 through 67563-15 show an oleic acid level ranging between about 34% (w/w) and about 50% (w/w) and a linoleic acid level ranging between about 7% (w/w) and about 18% (w/w). NIR analysis demonstrates that plants transformed with pMON67563 show an increase in total oil level and essentially the same protein level as compared with a control plant. Control strains 9979-11 through 9979-15 exhibit a total oil percentage ranging between about 33.5% and about 36.8%. Compared to the control strains, transformed strains 67563-1 through 67563-3 and 67563-5 through 67563-15 show an increased percentage of total oil and range from about 35.5% to about 38.9%. As illustrated by FIG. 2, when control and transformed strains are plotted to compare % total oil (x-axis) versus % oleic acid (18:1), an increase in oleic acid content is correlated with an increase in total oil content.

### Example 3

Arabidopsis plants transformed with pMON67563 (FIG. 1) are grown to the T₃ seed generation. T₃ seed is harvested and analyzed. Gas chromatography (GC) and near infrared (NIR) analysis are used to determine fatty acid profile and total oil content, respectively. Results of GC analyses demonstrate that 100% of progeny of the transformed plants have an increased level of oleic acid (18:1) similar to that observed for parent plants.

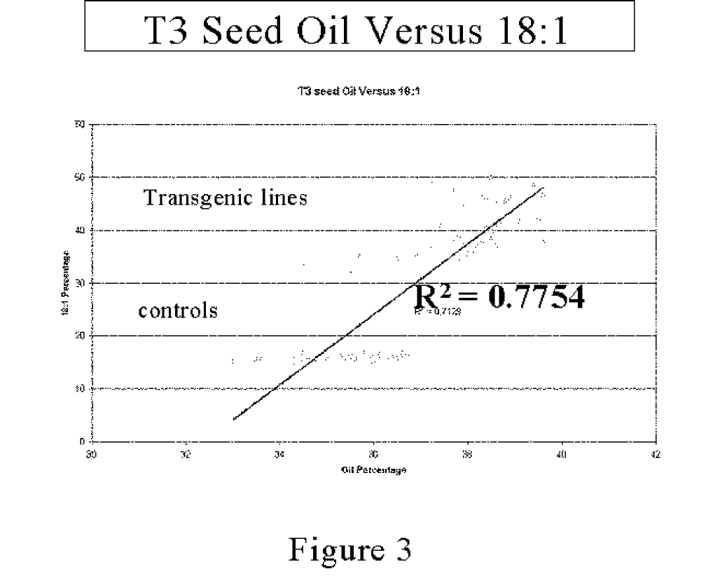

Progeny plants also exhibit an increase in total oil. A comparison of oleic acid (18:1) level versus percentage of total oil is provided in FIG. 3.

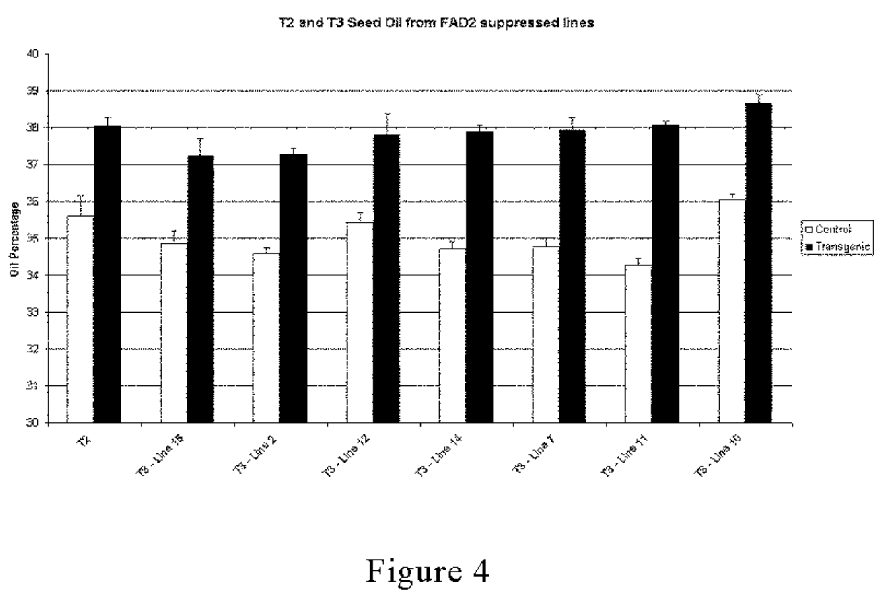

As illustrated in FIG. 4, mean oil percentage in T₂ and T₃ seed from transgenic lines is increased as compared to control seed containing an empty vector. The correlation between increased percent oleic acid and increased percent total oil evident in T₃ generation seeds appears to be genetically heritable.

As illustrated by FIG. 3, when control and transformed strains are plotted to compare percent total oil (x-axis) versus percent oleic acid (18:1), an increase in oleic acid content is correlated with an increased total oil content in transgenic Arabidopsis T₃ seed.

### Example 4

Canola FAD-2 constructA section of the Brassica napus FAD2 gene was isolated by PCR amplification. Primers 17942 (SEQ ID NO: 2) and 17944 (SEQ ID NO:3) were paired to amplify base pairs 284-781 of the FAD2 coding sequence from Brassica napus (cv. Ebony) genomic DNA. A NotI site was added to the 5′ end an NcoI site was added to the 3′ end of the fragment to facilitate cloning. The resulting PCR fragments were cloned into pCR2.1 Topo. The complete double strand sequence was obtained.

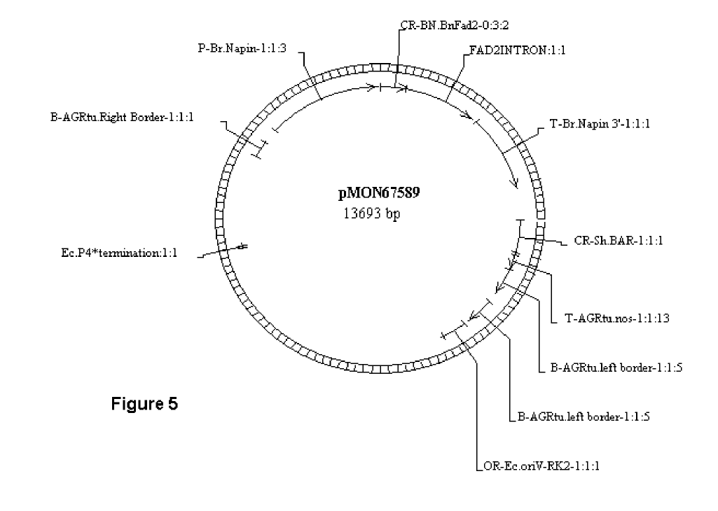

A 444 bp fragment containing CR-BN.BnFad2-0 (SEQ ID NO:4), was removed by digestion with NotI and NcoI. The fragment was ligated in between the Brassica napus promoter and first intron of the Arabidopsis FAD2 gene (At3g12120), which had been digested with NotI and NcoI. The resulting plasmid, was named pMON67589 (FIG. 5). The nucleic acid sequence was determined using known methodology and confirmed the integrity of the cloning junctions. A section of the Brassica napus FAD2 gene was isolated by PCR amplification. Primers 17943 (SEQ ID NO:5) and 17945 (SEQ ID NO:6) were paired to amplify base pairs 284-781 of the FAD2 coding sequence from Brassica napus (cv. Ebony) genomic DNA. A KpnI site was added to the 3′ end a SmaI site was added to the 5′ end of the fragment to facilitate cloning. The resulting PCR fragments were cloned into pCR2.1 Topo. The complete double strand sequence was obtained.

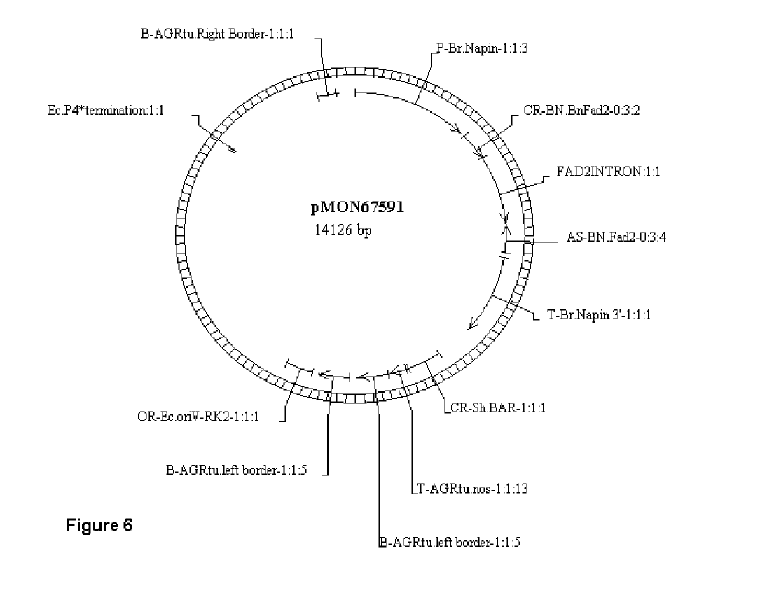

A 455 bp fragment containing AS-BN.BnFad2-0 (SEQ ID NO:7), was removed by digestion with KpnI and SmaI. The fragment was ligated in between the first intron of the Arabidopsis FAD2 gene (At3g12120) and napin 3′ UTR in pMON67589, which had been digested with SmaI and KpnI. The resulting plasmid, was named pMON67591 (FIG. 6). The nucleic acid sequence was determined using known methodology and confirmed the integrity of the cloning junctions.

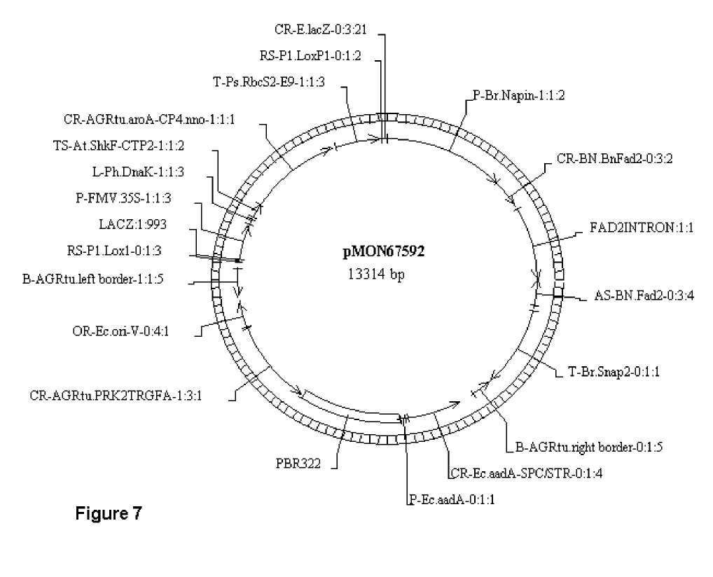

A 2030 bp fragment containing CR-BN.BnFad2-0 followed by the first intron of the Arabidopsis thaliana FAD2 gene (At3g12120) and AS-BN.BnFad2-0, was removed from pMON67591 by digestion with NotI and SmaI. The fragment was ligated into a plasmid that had been digested with NotI and HindIII (the HindIII site was blunt ended prior to ligation). The resulting plasmid was named pMON67592 (FIG. 7). The nucleic acid sequence was determined using known methodology and confirmed the integrity of the cloning junctions. This vector was used in the subsequent transformation of canola, which was done via Agrobacterium-mediated transformation.

### Example 5

Seeds from R2 canola plants transformed with pMON67592 were analyzed to determine total oil, oleic acid content and protein content. As can be seen in Table 1, differences between homozygous positive and null segregants ranged from 1.7-2.5% Total Oil and 20.4-25.6% oleic acid. Protein levels remained the same. Table 2 shows the combined results from all events.

TABLE 1
Average Total Oil and Oleic Acids Levels in R2 Canola seed derived from
five individual transformants.

| | | % Total OIL | | | | % Oleic Acid | | | |
|---|---|---|---|---|---|---|---|---|---|
| | | Homozygous | | Null Segregant | | Homozygous | | Null Segregant | |
| Event | N | Mean | Std Error | Mean | Std Error | Mean | Std Error | Mean | Std Error |
| BN_G1258 | 29 | 46.2 | 0.44 | 44.5 | 0.30 | 84.1 | 0.52 | 59.4 | 0.35 |
| BN_G1260 | 29 | 43.3 | 0.34 | 40.8 | 0.25 | 85.8 | 0.57 | 65.5 | 0.41 |
| BN_G1262 | 27 | 47.0 | 0.32 | 45.2 | 0.21 | 85.3 | 0.42 | 59.8 | 0.27 |
| BN_G1291 | 23 | 47.4 | 0.65 | 45.4 | 0.39 | 86.5 | 0.58 | 63.7 | 0.34 |
| BN_G1333 | 26 | 47.9 | 0.95 | 45.6 | 0.64 | 85.9 | 0.42 | 64.0 | 0.28 |

The mean and standard error were calculated in JMP Version: 4.0.4 (SAS Institute). The differences between means of homozygous positive and null segregants for both total oil and oleic acid for each of the 5 events is statistically significant (p < .0001)

TABLE 2
Average Total Oil and Oleic Acid Levels in R2 Canola seed
transformed with pMON65792

| | % TOTAL OIL | | | % OLEIC ACID | | |
|---|---|---|---|---|---|---|
| Zygosity | N | Mean | StDev | N | Mean | StDev |
| Homozygous | 94 | 44.93 | 2.74 | 51 | 85.16 | 1.52 |
| Null Segregant | 178 | 42.98 | 2.33 | 123 | 63.72 | 3.39 |
| Difference | | 1.95 | | | 21.4 | |

The mean and standard deviation were calculated in JMP Version: 4.0.4 (SAS Institute). Plants were derived from 5 independent transformants. The differences between means of homozygous positive and null segregants is statistically significant (p < .0001)

### Example 6

On the basis of sequence similarity to Arabidopsis, soy and maize delta-12 desaturases (FAD2), four genes were identified in a proprietary corn unigene data base. They have been designated FAD2-1, FAD2-2, FAD2-3 and FAD2-4. The full-length cDNA sequence of Zm. FAD2-1 is shown in SEQ ID NO:8. It encodes a polypeptide of 387 amino acids (translation frame: nucleotide 182-1342). The full-length cDNA sequence of Zm. FAD2-2 is shown in SEQ ID NO:9. It encodes a polypeptide of 390 amino acids (translation frame: nucleotide 266-1435).The full-length cDNA sequence of Zm. FAD2-3 is shown in SEQ ID NO:10. It encodes a polypeptide of 382 amino acids (translation frame: nucleotide 170-1315). The partial sequence of Zm. FAD2-4 is shown in SEQ ID NO:11. It encodes a partial polypeptide of 252 amino acids (translation frame: nucleotide 1-256).

The coding regions of the three genes share significant sequence identity. FAD2-1 shares 91% identity to FAD2-2at the nucleotide level and 88% identity at the amino acid level. FAD2-1 shares 85% identity to FAD2-3 at the nucleotide level and 68% identity at the amino acid level. FAD2-1 shares 82% identity to FAD2-4 at the nucleotide level and 68% identity at the amino acid level. FAD2-3 shares 80% identity to FAD2-4 at the nucleotide level and 65% identity at the amino acid level.

A virtual northern was used to determine which of the 4 genes were present in the seed tissue of corn. Both FAD2-1 and FAD2-2 were present in whole seeds, germ tissue and embryo tissue collected at different times during seed development. Neither FAD2-3 nor FAD2-4 were present in the seed tissues but both were detected in leaf tissue.

RNAi construct from a fusion of 3′UTR of FAD2-1 and FAD2-2

An expression construct comprising a corn L3 promoter, a rice-actin intron 3′ to the promoter and 5′ to the RNAi element, an RNAi element followed by a globulin 3′end located 3′ to the RNAi element was constructed. The RNAi element was composed of a fragment of the Zm. FAD2-1 3′UTR joined by a BamH1 site to a fragment of the Zm. FAD2-2 3′UTR both in the sense orientation linked to the same two FAD2 3′UTR fragments in the antisense orientation by an HSP70 intron containing intron splice sites. The HSP70 intron is located such that it is in the sense orientation relative to the promoter. The order of sense and antisense of the 3′UTR fragments is not important as long as each fragment (FAD2-1 and FAD2-2) is sense on one side of the center intron and antisense on the other. The construct is suitable for transformation into corn either by microprojectile bombardment or by Agrobacterium-mediated transformation.

PCR was used to obtain the HSP70 intron with a Bsp120I site on the 5′ end and a Stu1 site on the 3′ end. Primers (SEQ ID NOS:12 and 13) specific for the HSP70 intron sequence were used to clone the intron.

The Bsp120I and StuI fragment of the 820 base pair PCR product (SEQ ID NO:14) was cloned into the same sites of a turbo binary containing a cauliflower mosaic virus promoter driving nptII with a NOS 3′ and a Zea mays L3 promoter followed by a rice actin intron and a globulin 3′ to make an intermediate construct.

The fragments of the Zm. FAD2-1 and FAD2-2 3′UTRs were obtained by PCR. Monsanto library clones were used as templates with primers specific for FAD2-1 (SEQ ID NO:15, containing added cloning sites Sse83871 and Sac1; and SEQ ID NO:16, containing an added cloning site BamH1) or primers specific for FAD2-2 (SEQ ID NOS:17, containing an added cloning site BamH1; and SEQ ID NO:18, containing added sites Bsp120I and EcoRV).

To link the two PCR products, they were each digested with BamH1, gel purified, ligated and the ligation product used as a template with primers SEQ ID NOS:15 and 18. The resulting 447 base pair fragment (SEQ ID NO:19).

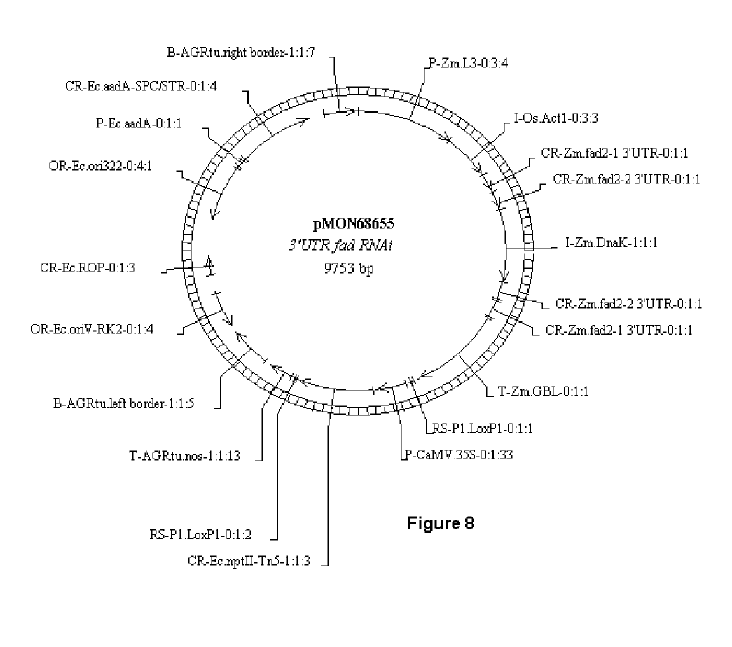

The Sac1/Bsp120I fragment of SEQ ID NO:19 was cloned into the same sites and the Sse8387I/EcoRV fragment of SEQ ID NO:19 is cloned into the Sse83871/Stu1 sites of the intermediate construct to produce pMON56855 (FIG. 8).

### Example 7

RNAi construct from a fusion of introns of FAD2-1 and FAD2-2

An expression construct comprising a corn L3 promoter, a corn rice-actin intron 3′ to the promoter and 5′ to the RNAi element, an RNAi element followed by a globulin 3′end located 3′ to the RNAi element was constructed. The RNAi element was composed of a portion of the Zm. FAD2-1 intron joined by a BamH1 site to a portion of the Zm. FAD2-2 intron both in the sense orientation linked to the same two FAD2 intron fragments in the antisense orientation by an HSP70 intron containing intron splice sites. The HSP70 intron is located such that it is in the sense orientation relative to the promoter. The order of sense and antisense of the intron fragments is not important as long as each fragment (FAD2-1 and FAD2-2) is sense on one side of the center intron and antisense on the other. The construct is suitable for transformation into corn either by microprojectile bombardment or by Agrobacterium-mediated transformation.

PCR was used to obtain the HSP70 intron as described in the previous example. Fragments from introns from the Zm. FAD2-1 and FAD2-2 genes were obtained by PCR. Genomic DNA prepared from the leaves of Z. mays variety LH59 using the protocol of Dellaporta et al. (Dellaporta et al. (1983) A plant DNA minipreparation: version II. Plant Mol Biol Rep 1: 19-21) was used as the template. For FAD2-1, specific primers (SEQ ID NO:20, with added cloning sites Sse83871 and Sac1; and SEQ ID NO:21) were used to produce a 267 base pair product (SEQ ID NO:22). For FAD2-2, specific primers (SEQ ID NO:23, which included 21 bases that overlap with the 3″ sequence of SEQ ID NO:22; and SEQ ID NO:24, containing added sites Bsp120I and EcoRV) were used to produce a 260 base pair product (SEQ ID NO:25).

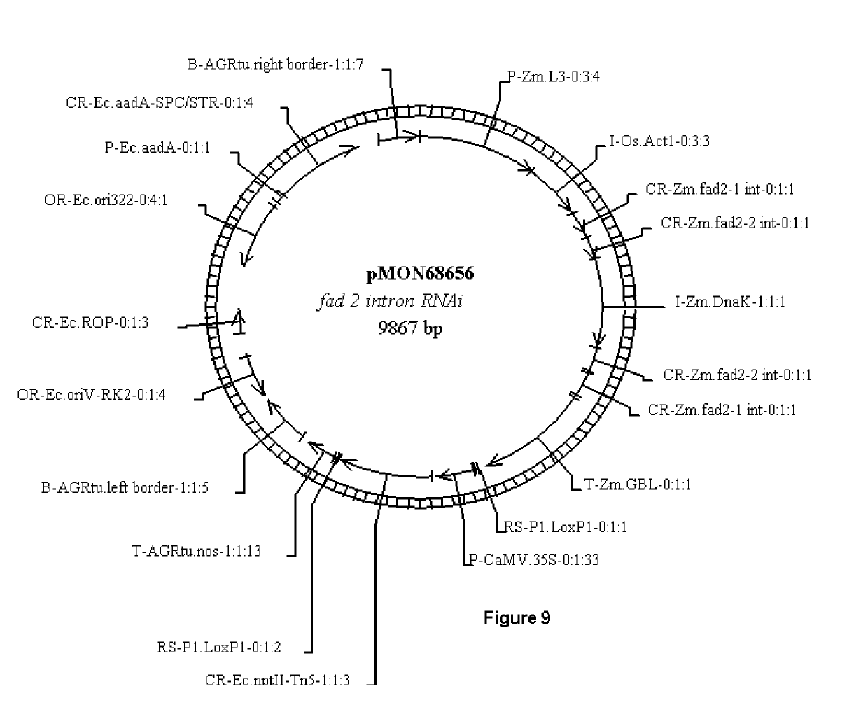

To link the two PCR products (SEQ ID NOS:22 and 25), they were both used as templates in a PCR reaction using primers SEQ ID NO:20 and SEQ ID NO:24 to produce a 506 base pair fusion (SEQ ID NO:26). The Sac1 and Bsp 120I fragment from SEQ ID NO:26 was gel purified then cloned into the same sites to produce pMON68656 (FIG. 9).

1. A method for increasing total oil level in a seed comprising:
(A) transforming a plant with a nucleic acid construct that comprises as operably linked components, a promoter, a structural nucleic acid sequence capable of modulating the level of FAD2 mRNA or FAD2 protein; and
(B) growing said plant.

2. The method for increasing total oil level in a seed according to claim 1, wherein said plant is Arabidopsis.

3. The method for increasing total oil level in a seed according to claim 1, wherein said plant is corn.

4. The method for increasing total oil level in a seed according to claim 1, wherein said plant is canola.

5. The method for increasing total oil level in a seed according to claim 1, wherein said promoter is a seed specific promoter.

6. The method for increasing total oil level in a seed according to claim 5, wherein said seed specific promoter is selected from the group consisting of napin promoter, soybean trypsin inhibitor promoter, ACP promoter, stearoyl-ACP desaturase promoter, soybean a′ subunit of b-conglycinin promoter, oleosin promoter, β-conglycinin promoter, maize globulin-1 gene promoter, and zein promoter.

7. The method for increasing total oil level in a seed according to claim 1, wherein the level of total protein remains essentially unchanged in said seed as compared to a seed from a second plant lacking said nucleic acid construct.

8. The method for increasing total oil level in a seed according to claim 1, wherein the level of oleic acid is increased and the level of linoleic acid is decreased in said seed as compared to a seed from a second plant lacking said nucleic acid construct.

9. The method for increasing total oil level in a seed according to claim 1, wherein the percentage of total oil in said seed is increased as compared to a seed from a second plant lacking said nucleic acid construct.

10. A method for increasing total oil in a seed comprising:
(A) transforming a plant with a nucleic acid construct that comprises as operably linked components, a promoter, a structural nucleic acid sequence capable of increasing the level of oleic acid; and
(B) growing said plant.

11. A chimeric gene comprising the nucleic acid fragment selected from the group consisting of SEQ ID NOS: 1, 4, 7-11, 14, 19, 22, 25 and 26 or the reverse complement thereof, any functionally equivalent subfragment thereof or the reverse complement of said fragment or subfragment wherein said fragments are operably linked and further wherein expression of the chimeric gene results in an increase in total oil.

12. A method for increasing total oil level in a seed comprising:
(A) transforming a plant with a nucleic acid construct that comprises as operably linked components, a promoter, a sequence selected from the group consisting of SEQ ID NOS: 1, 4, 7-11, 14, 19, 22, 25 and 26 or the reverse complement thereof, any functionally equivalent subfragment thereof or the reverse complement of said fragment or subfragment; and
(B) growing said plant.

13. The method for increasing total oil level in a seed according to claim 12, wherein said plant is Arabidopsis.

14. The method for increasing total oil level in a seed according to claim 12, wherein said plant is corn.

15. The method for increasing total oil level in a seed according to claim 12, wherein said plant is canola.

16. The method for increasing total oil level in a seed according to claim 12, wherein said promoter is a seed specific promoter.

17. The method for increasing total oil level in a seed according to claim 16, wherein said seed specific promoter is selected from the group consisting of napin promoter, soybean trypsin inhibitor promoter, ACP promoter, stearoyl-ACP desaturase promoter, soybean a′ subunit of b-conglycinin promoter, oleosin promoter, β-conglycinin promoter, maize globulin-1 gene promoter, and zein promoter.

18. The method for increasing total oil level in a seed according to claim 13, wherein the level of total protein remains essentially unchanged in said seed as compared to a seed from a second plant lacking said nucleic acid construct.

19. The method for increasing total oil level in a seed according to claim 13, wherein the level of oleic acid is increased and the level of linoleic acid is decreased in said seed as compared to a seed from a second plant lacking said nucleic acid construct.

20. The method for increasing total oil level in a seed according to claim 13, wherein the percentage of total oil in said seed is increased as compared to a seed from a second plant lacking said nucleic acid construct.


SEQUENCE LISTING (26 sequences)

SEQ ID NO: 1 - DNA, 120 bp, Arabidopsis thaliana
gcatgatggtgaagaaattgtcgacctttctcttgtctgtttgtcttttgttaaagaagctatgcttcgttttaataatcttattgtccattttgttgtgttatgacattttggctgctc

SEQ ID NO: 2 - DNA, 31 bp, Artificial
  primer
gcggccgcgcgtcctaaccggcgtctgggtc

SEQ ID NO: 3 - DNA, 28 bp, Artificial
  primer
ccatgggagaccgtagcagacggcgagg

SEQ ID NO: 4 - DNA, 440 bp, Brassica napus
gcgcgtcctaaccggcgtctgggtcatagcccacgagtgcggccaccacgccttcagcgactaccagtggcttgacgacaccgtcggtctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggccgttgtacttagccttcaacgtctcgggaagaccttacgacggcggcttcgcttgccatttccaccccaacgctcccatctacaacgaccgcgagcgtctccagatatacatctccgacgctggcatcctcgccgtctgctacggtctcc

SEQ ID NO: 5 - DNA, 29 bp, Artificial
  primer
cccggggcgtcctaaccggcgtctgggtc

SEQ ID NO: 6 - DNA, 28 bp, Artificial
  primer
ggtaccgagaccgtagcagacggcgagg

SEQ ID NO: 7 - DNA, 441 bp, Brassica napus
cgagaccgtagcagacggcgaggatgccagcgtcggagatgtatatctggagacgctcgcggtcgttgtagatgggagcgttggggtggaaatggcaagcgaagccgccgtcgtaaggtcttcccgagacgttgaaggctaagtacaacggccagccgagagtgaactgaaccgttaacatcacggtgcgtcccaaagggttgttgaggtacttgccgtaccacttgatgtctgacttcttcttggggacaaacacttcgtctctctcgagggagccagtgttggaatggtggcgtcgatgactgtacttccaggagaagtaagggacgaggaggaaggagtggaagatgagaccgacggtgtcgtcaagccactggtagtcgctgaaggcgtggtggccgcactcgtgggctatgacccagacgccggttaggacgcccc

SEQ ID NO: 8 - DNA, 1729 bp, Zea mays
ctgcagacaccaccgctcgtttttctctccgggacaggagaaaaggggagagagaggtgaggcgcggtgtccgcccgatctgctctgccccgacgcagctgttacgacctcctcagtctcagtcaggagcaagatgggtgccggcggcaggatgaccgagaaggagcgggagaagcaggagcagctcgcccgagctaccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactactcgctcctggacgacgtggtcggcctggtgctgcactcgtcgctcatggtgccctacttctcgtggaagtacagccaccggcgccaccactccaacacggggtccctggagcgcgacgaggtgttcgtgcccaagaagaaggaggcgctgccgtggtacaccccgtacgtgtacaacaacccggtcggccgggtggtgcacatcgtggtgcagctcaccctcgggtggccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtacaagctggcggcggcgttcggggtctggtgggtggtgcgcgtgtacgccgtgccgctgctgatcgtgaacgcgtggctggtgctcatcacctacctgcagcacacccacccgtcgctcccccactacgactcgagcgagtgggactggctgcgcggcgcgctggccaccatggaccgcgactacggcatcctcaaccgcgtgttccacaacatcacggacacgcacgtcgcgcaccacctcttctccaccatgccgcactaccacgccatggaggccaccaaggcgatcaggcccatcctcggggactactaccacttcgacccgacccctgttgccaaggcgacctggcgcgaggccagggagtgcatctacgtcgagcccgaggaccgcaagggcgtcttctggtacaacaagaagttctagccgccgccgctcgcagagctgagaggacgctaccataggaatgggagcaggaaccaggaggaggagacggtactcgccccaaagtctccgtcaacctatctaatcgttagtcgtcagtcttttagacgggaagagagatcatttgggcacagagacgaaggcttactgcagtgccatcgctagagctgccatcaagtacaagtaggcaaattcgtcaacttagtgtgtcccatgttgtttttcttagtcgtccgctgctgtaggctttccggcggcggtcgtttgtgtggttggcatccgtggccatgcctgtgcgtgcgtggccgcgcttgtcgtgtgcgtctgtcgtcgcgttggcgtcgtctcttcgtgctccccgtgtgttgttgtaaaacaagaagatgttttctggtgtctttggcggaataaaaa

SEQ ID NO: 9 - DNA, 1804 bp, Zea mays
ccgaaccgaggcggccaggctccctcctccctcctcctccctgcaaatcgccaaatcctgcaggcaccaccgctcgttttcctgtgcggggaacaggagagaaggggagagaccgagagagggggaggcgcggcgtccgccggatctgctccgacccccgacgcagcctgtcacgccgtcctcactctcagccagcgaaaatgggtgccggaggcaggatgaccgagaaggagcgggaggagcaggagcaagtcgcccgtgctaccggcggtggcgcggcagtgcagcggtcgccggtggagaagccgccgttcacgttggggcagatcaagaaggcgatcccgccgcactgcttcgagcgctccgtgctgaggtccttctcctacgtggcccacgacctggcgaccgccgcggcgctcctctacctcgcggtggccgtgataccggcgctacccagcccgctccgctacgcggcctggccgctgtactgggtggcccaggggtgcgtgtgcacgggcgtgtgggtgatcgcgcacgagtgcggccaccacgccttctccgaccacgcgctcctggacgacgccgtcggcctggcgctgcactcggcgctgctggtgccctacttctcgtggaagtacagccaccggcgccaccactccaacacggggtccctggagcgcgacgaggtgttcgtgccgaggaccaaggaggcgctgccgtggtacgccccgtacgtgcacggcagccccgcgggccggctggcgcacgtcgccgtgcagctcaccctgggctggccgctgtacctggccaccaacgcgtcgggccgcccgtacccgcgcttcgcctgccacttcgacccctacggcccgatctacggcgaccgggagcgcgcccagatcttcgtctcggacgccggcgtcgcggccgtggcgttcgggctgtacaagctggcggcggcgttcgggctctggtgggtggtgcgcgtgtacgccgtgccgctgctgatcgtcaacgcgtggctggtgctcatcacgtacctgcagcacacccacccggcgctgccccactacgactcgggcgagtgggactggctgcgcggcgcgctcgccaccgtcgaccgcgactacggcgtcctcaaccgcgtgttccaccacatcacggacacgcacgtcgcgcaccacctcttctccaccatgccgcactaccacgccgtggaggccaccagggcgatcaggcccgtcctcggcgactactaccagttcgacccgacccctgtcgccaaggccacctggcgcgaggccagggagtgcatctacgtcgagcctgagatccgcaacagcaagggcgtcttctggtacaacagcaagttctagccgccgcttgctttttccctaggaatgggaggagaaatcaggatgagaagatggtaatgtctccatctacctgtctaatggttagtcaccagtctttagacaggaagagagcatttgggcttcagaaaaggaggcttactgcactactgcagtgccatcgctagatctaggcaaattcagtgtgtctgtgcccatggctgtgagctttgggtactctcaagtagtcaagttctcttgtttttgtttttagtcgtcgctgttgtaggcttgccggcggcggccgttgcgtggccgcgccttgtcgtgtgcgtcttgcttttgtgtgcgttcgtgctcccttgtttttgtgtgcgttcgtgctcccttcgtgttgttgtaaaacactagtctggtgtctttggcggaataactaacagatcgtcgaacgaaaa

SEQ ID NO: 10 - DNA, 1543 bp, Zea mays
cctgcaggtaccggtccggaattcccgggtcgacccacgcgtccgcatcctcaaagcctccggttgcccgaagcagtcgcatctgctcttcgtggcaccgaactcttggagcaatcaacttttgaatcgtcgacaggacagccgcgcgcgtcgtggcgaaggctgcaggatggagcagcagacgaagacgacgacacagcaagagggcaaaggcctcgccaccatggagcggtcgatcgtggacaagccgccattcacgctagcggacctcaggaaggccatcccgccgcactgcttccagcgctcgctcatcaggtcctgctcctacctcgcccacgacctcgccatcgccgcggggctcctgtacttggctctggccgtcatccccgccctcccgggcgtcctcctccgcgccgccgcctggccgctctactgggcggcgcagggcagcatcatgttcggcgtgtgggtgatcgcgcacgagtgcgggcacagcagcttctcccgctacggcctcctcaacgacgccctcggcctggtgctgcactcgtgcctcttcgcgccctacttctcgtggaagtacagccaccagcgccaccacgccaacaccgcgtccctggagcgcgacgaggtgttcgtgcccaagcagaggcccgagatgccgtggtactccccgctcgtgtacaagcgcgacaaccccgtcgcccggctggtcctcctcgccgtgcagctcaccgtcggctggcccatgtacctggcgttcaacacctggggccgccgctactcccgcttcgcgtgccacttcgacccctacagccccatctacggcgaccgggagcgcgcccagatcgccgtctccgacgccggcgtcctggccgtgtcgttcgcgctgtacaggctcgccgcggcccacgggctctggcccgtggtcagcgtctacggcgtgccgctgctggtgacgaacgcctggctcgtggtggtcacgtacctgcaccacacgcaccgcgcgctcccgcactacgactccagcgagtgggactggatgcgcggggcgctcgccaccgtcgaccgcgactacggcgtcctcaaccgcgtgttccaccacatcgccgacacgcatatcgctcaccatctcttcccggccattccgcactaccacgccatggaggccaccagagcgatccgtcctgtcctcggcgactactaccgctccgatagcacgcccatagccgaggcgctctggcgcgaggctaaagagtgcatctacgtccagcgcgacgaccagaagggcgtattttggtacaagaacgtgttctagctgcagagctgctggacgacgcaaaccccgagcggagccataggggcacagaaataatattatttgtggtcttgtacattttgttatatatttaccttgcacatgtcacaaataaaaaactggcatatatatataacaaaatgtatactatacgtatatatatgtatcatcttgtgttatatgttaaatgtttaagatgttttaaatgccaaaaaaaaaaaaaaa

SEQ ID NO: 11 - DNA, 774 bp, Zea mays
ctgcaggtaccggtccggaattcccgggtcgacccacgcgtccgagcctctcgctgtgcattgaccagcgcagagacaagtagagcagggagggaagcccatcgtgtgtttctcagtcccagtcagcagcatggctgccggcgtcgcaacggcggaggagatcaggaagaagagccactcgggcggtgtgcggcggtcgccggtggacaggccgccgttcacgctgggggacatcaagagggccatcccgccgcactgcttccagcgctcggcgctcaggtccttctcgtacctcctccacgacctcgccatcgcggccgggctcctgtacctggccgtggcgggcatcccggcgctcccgagcgccgcgctccgccgcttcgtggcgtggccgctctactgggcggcgcagggcagcgtgctgacgggcgtctgggtcatcgggcacgagtgcggccaccacgccttctccgactacccgctcctggacaacgccgtcggcttcgtgctccactccgcgctgctcacgcccttcttcgcctggaagtacagccaccggcgccaccacgccaacaccggctccatggagaacgacgaggtgtacgtggccaagacccgggacgcgctgcggtggtacacgccgctcgtgttcggcaacccggtcggccggctggtgtacatcgcgctgcagctcaccctcgcgtggccgctctacctggcgttcaacctctcagggcagaactacggcggccgctctagaggatccaagcttac

SEQ ID NO: 12 - DNA, 29 bp, Artificial
  primer
ttgggcccaccgtcttcggtacgcgctca

SEQ ID NO: 13 - DNA, 28 bp, Artificial
  primer
gcaggcctccgcttggtatctgcattac

SEQ ID NO: 14 - DNA, 820 bp, Zea mays
ttgggcccaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggaggcctgc

SEQ ID NO: 15 - DNA, 34 bp, Artificial
  primer
cctgcaggagctcagagctgagaggacgctacca

SEQ ID NO: 16 - DNA, 28 bp, Artificial
  primer
gtggatccactaagttgacgaatttgcc

SEQ ID NO: 17 - DNA, 30 bp, Artificial
  primer
gtggatccgtgtgtctgtgcccatggctgt

SEQ ID NO: 18 - DNA, 35 bp, Artificial
  primer
cgatatcgggcccgtgttttacaacaacacgaagg

SEQ ID NO: 19 - DNA, 447 bp, Zea mays
cctgcaggagctcagagctgagaggacgctaccataggaatgggagcaggaaccaggaggaggagacggtactcgccccaaagtctccgtcaacctatctaatcgttagtcgtcagtcttttagacgggaagagagatcatttgggcacagagacgaaggcttactgcagtgccatcgctagagctgccatcaagtacaagtaggcaaattcgtcaacttagtggatccgtgtgtctgtgcccatggctgtgagctttgggtactctcaagtagtcaagttctcttgtttttgtttttagtcgtcgctgttgtaggcttgccggcggcggccgttgcgtggccgcgccttgtcgtgtgcgtcttgcttttgtgtgcgttcgtgctcccttgtttttgtgtgcgttcgtgctcccttcgtgttgttgtaaaacacgggcccgatatcg

SEQ ID NO: 20 - DNA, 32 bp, Artificial
  primer
cctgcaggagctctgtgatccccaacttgctg

SEQ ID NO: 21 - DNA, 24 bp, Artificial
  primer
ctgacacaaacgaggaagtacgct

SEQ ID NO: 22 - DNA, 267 bp, Zea mays
cctgcaggagctctgtgatccccaacttgctgtggcgtggtagttggatcgtgtttaggcaagaaagtaaatgcgatcatgcacggcatatttgccaccttcctgggagacgccccctcgtgccgtgatctgttttactttggttgattggtggcctttctcgtggttcacgtgacagcttttctgatgggatgagatcactgtaatgttgttgcttgattcacgctcgcttgatcttactgtagcgtacttcctcgtttgtgtcag

SEQ ID NO: 23 - DNA, 36 bp, Artificial
  primer
gtacttcctcgtttgtgtcaggcaagaaagtgatgc

SEQ ID NO: 24 - DNA, 32 bp, Artificial
  primer
cgatatcgggcccattttcgctggttgctggc

SEQ ID NO: 25 - DNA, 260 bp, Zea mays
gtacttcctcgtttgtgtcaggcaagaaagtgatgcggtcgtgcacggcacatgccagctttgtgggagccgcccctaaccctcgctgaatcagtcagtagtgccaacttgctagagttttttttcttcttgttttggttcactcgacagatttttgtttggatgagatcgctgcaacattgttcttgatccacacttgcctgatcttaccgtctcgttcgtgttcgtgccagcaaccagcgaaaatgggcccgatatcg

SEQ ID NO: 26 - DNA, 506 bp, Zea mays
cctgcaggagctctgtgatccccaacttgctgtggcgtggtagttggatcgtgtttaggcaagaaagtaaatgcgatcatgcacggcatatttgccaccttcctgggagacgccccctcgtgccgtgatctgttttactttggttgattggtggcctttctcgtggttcacgtgacagcttttctgatgggatgagatcactgtaatgttgttgcttgattcacgctcgcttgatcttactgtagcgtacttcctcgtttgtgtcaggcaagaaagtgatgcggtcgtgcacggcacatgccagctttgtgggagccgcccctaaccctcgctgaatcagtcagtagtgccaacttgctagagttttttttcttcttgttttggttcactcgacagatttttgtttggatgagatcgctgcaacattgttcttgatccacacttgcctgatcttaccgtctcgttcgtgttcgtgccagcaaccagcgaaaatgggcccgatatcg